(19) United States
(12) Patent Application Publication
(10) Pub. No.: US 2025/0110097 A1
(43) Pub. Date: Apr. 3, 2025

(54) METHOD FOR EVALUATING HOMOGENEITY OF ORAL SUSPENSIONS

(71) Applicant: Kenvue Brands LLC, Summit, NJ (US)

(72) Inventors: Austin Courtney Suder, Downingtown, PA (US); Lindsay M. Vieth, Willow Grove, PA (US); Jonathan Zeszotarski, Ambler, PA (US)

(21) Appl. No.: 18/892,968

(22) Filed: Sep. 23, 2024

Related U.S. Application Data
(60) Provisional application No. 63/586,459, filed on Sep. 29, 2023.

(51) Int. Cl. G01N 33/15

(52) U.S. Cl. CPC G01N 33/15

(57) ABSTRACT

A method for evaluating homogeneity of a suspension containing an active agent, including the steps of obtaining a test sample of the suspension in a container; subjecting the container to shaking at a shaker speed and for a duration of shaking; removing an aliquot of the suspension from the container; measuring the amount of the active agent in the aliquot; and comparing the amount of the active agent in the aliquot to an acceptance criteria. In one example, the suspension is a pharmaceutical suspension and the active agent is substantially insoluble.

### CROSS-REFERENCE TO RELATED APPLICATIONS

This application claims the benefit of U.S. provisional application 63/586,459 filed on Sep. 29, 2023, the complete disclosure of which is hereby incorporated herein by reference for all purposes.

### FIELD OF THE INVENTION

The present invention relates to methods for evaluating the homogeneity of oral suspensions containing an active agent, especially as related to oral suspensions used as over-the-counter pharmaceutical products.

### BACKGROUND OF THE INVENTION

Orally administered medicaments or pharmaceuticals are given to the patient in many forms, including solid forms, such as, capsules, caplets, gel caps, or tablets, and liquid forms, such as, solutions, e.g., syrups and elixirs, emulsions, or suspensions. For many patients, including pediatric and geriatric patients, a liquid oral dosage form is preferable over solid dosage forms because of the ready swallowability without the need to chew and/or swallow a solid dosage form. Liquid dosage forms have been on the market for over-the-counter (“OTC”) pharmaceutical products for decades.

One type of liquid dosage form is an oral suspension, also known as a pharmaceutical suspension. Suspensions are generally understood to be a two-phase system having solid substantially insoluble active agent particles dispersed throughout liquid medium. For example, the active agent may be substantially water insoluble. The challenges of keeping the substantially insoluble active agent suspended, assuring stability of the substantially insoluble active agent, and maintaining dose uniformity for a prolonged period of time, have been previously addressed. See for example, U.S. Pat. Nos. 5,409,907, and 5,374,659.

However, the solutions to such challenges must be re-evaluated when OTC suspension products are formulated or re-formulated to use reduced ingredients or naturally sourced ingredients. The term “clean” or “clean medicine” is a newly emerging trend in OTC pharmaceutical products. Although there is no generally accepted definition for the term “clean”, it is generally understood by consumers to mean any of the following: no artificial flavors, dyes, preservatives or sweeteners; sourced from nature; and/or no artificial, chemical or unnecessary ingredients.

Formulating “clean” oral suspensions may limit or exclude certain ingredients that have been traditionally used in pharmaceutical suspensions to overcome the challenges of keeping the substantially insoluble active agents suspended, assuring stability of the substantially insoluble active agents, and maintaining dose uniformity for a prolonged period of time. Newly formulated pharmaceutical suspension formulations, including those that fit the definition of “clean,” must be evaluated to ensure that they meet the safety and efficacy standards that are required of all OTC pharmaceutical products.

Traditional pharmaceutical suspensions, which often remain uniformly suspended throughout their shelf life, may include the precautionary instruction to “shake well” on their instructions for use. User instruction(s), also referred to herein as shaking instruction(s), are even more critical for pharmaceutical suspension products that have “clean” ingredients that may cause natural separation of the product during non-use. In some cases, the natural separation of the “clean” product may be high or extreme enough to be visually apparent to the consumer. In other cases, especially if the bottle or packaging is not transparent, the natural separation of the product may not be visible to the consumer. Especially where there may be toxicity concerns related to exceeding the recommended dose of a particular active pharmaceutical ingredient (“API”), it is important to confirm that pharmaceutical suspensions that do need to be shaken prior to use will be safe and effective for consumers, and that instructions to shake are clearly understood and attainable.

The present invention provides new methodologies to evaluate the homogeneity of oral suspensions to ensure that such oral suspensions, especially pharmaceutical suspensions, will be safe and effective for consumer use. Such methodologies may be used during the product development process, and may further be used to verify that product instructions for product use are accurate and reproducible. Such methodologies may also be used for quality control purposes, stability testing, or even as part of a marketing demonstration or consumer testing protocol.

### SUMMARY OF THE INVENTION

The present invention may relate to a method for evaluating homogeneity of a suspension containing an active agent, including the steps of: (a) obtaining a test sample of the suspension in a container; (b) subjecting the container to shaking at a shaker speed and for a duration of shaking; (c) removing an aliquot of the suspension from the container; (d) measuring the amount of the active agent in the aliquot; and (e) comparing the amount of the active agent in the aliquot to an acceptance criteria.

According to an example, the shaker speed may be a low speed of about 140 rpm to about 220 rpm, or about 160 rpm to about 200 rpm, or about 170 rpm to about 190 rpm, or about 175 rpm to about 185 rpm, or about 180 rpm. According to another example, the shaker speed may be a high speed of about 240 rpm to about 320 rpm, or about 260 rpm to about 300 rpm, or about 270 rpm to about 290 rpm, or about 275 rpm to about 285 rpm, or about 280 rpm.

According to an example, the duration of shaking may be about 5 seconds to about 45 seconds, or about 5 seconds, or about 10 seconds, or about 15 seconds, or about 30 seconds or about 45 seconds.

According to an example, the method may include shaking the container along a vertical axis. According to another example, the method may include shaking the container along a horizontal axis. According to an example, the method may include shaking the container at an angle to a vertical axis, for example at an angle that is about 10 degrees to about 80 degrees from the vertical axis.

According to an example, the shaking may be conducted using a fixed speed reciprocal shaker.

According to an example, the test sample may be about 4 or 8 fluid ounces.

According to an example, the aliquot may be removed using a pipette. According to an example, the aliquot may be removed from a top portion of the container. According to an example, the aliquot may be removed from a bottom portion of the container. According to an example, the aliquot may be removed from a middle portion of the container.

According to another example, the aliquot may be removed using a syringe.

According to an example, the container may be inverted prior to removal of the aliquot. According to an example, the aliquot may be poured or squeezed from the container into a dosing cup.

According to an example, the active agent is substantially insoluble.

According to an example, the active agent may be an active pharmaceutical ingredient (API). According to an example, the amount of the active agent in the aliquot may be measured using an assay analysis of a USP monograph related to the API. According to an example, the acceptance criteria may correspond to an approved dose of the API.

According to an example, the method may further include the steps of: (f) subjecting a remaining test sample of the suspension in the container to additional shaking at the shaker speed and for a second duration of shaking; (g) removing a second aliquot of the suspension from the container; (h) measuring the amount of the active agent in the second aliquot; and (i) comparing the amount of the active agent in the second aliquot to a second acceptance criteria. According to an example, the second acceptance criteria may be the same as the acceptance criteria. According to another example, the second acceptance criteria may be different from the acceptance criteria.

According to one example, the step of subjecting the container to shaking at the shaker speed and for the duration of shaking may correspond to a user instruction associated with the test sample. Such user instruction may be provided on a commercial package of the test sample, for example to “shake well.”

According to another example, the step of subjecting the container to shaking at the shaker speed and for the duration of shaking may correspond to an observation of a user based on the user's interaction with a user instruction associated with the test sample. Such user instruction may be provided on a commercial package of the test sample, for example to “shake well.” In one example, the observation may be the user's shaker speed and duration of shaking.

### BRIEF DESCRIPTION OF THE DRAWINGS

FIGS. 1A-1E depict different orientations of a bottle containing a test suspension for shaking according to the methodologies of the present invention.

FIGS. 2A-2C depict different sampling areas of a bottle containing a test suspension according to the methodologies of the present invention.

### DETAILED DESCRIPTION OF THE INVENTION

It is believed that one skilled in the art can, based upon the description herein, utilize the present invention to its fullest extent. The following specific examples are to be construed as merely illustrative, and not as limiting the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified. In addition, all ranges set forth herein are meant to include any combinations of values between the two endpoints, inclusively.

As used herein, the term “active ingredient” or “active agent” is used herein in a broad sense and may encompass any material that imparts a therapeutic effect. For example, the active ingredient can be a pharmaceutical, nutraceutical, vitamin, dietary supplement, nutrient, herb, foodstuff, dyestuff, nutritional, mineral, supplement, oral care agent or flavoring agent (sensate) or the like and combinations thereof.

“Aliquot” refers to a sample taken from a larger whole for chemical analysis or other treatment.

“Dose” refers to the specific pre-determined amount of a certain ingredient, and for example, an active ingredient to be administered to a subject. The dose of particular active ingredients may be regulated by the United States Food and Drug Administration (FDA) or other regulatory bodies.

“Dosage form” applies to any composition designed to contain a dose of a certain ingredient, and for example, an active ingredient as defined herein. Suitable dosage forms may be pharmaceutical drug delivery systems and systems for delivering minerals, vitamins and other nutraceuticals, oral care agents, flavorants, and the like. In a particularly preferred example, the dosage form is an orally administered system for delivering a pharmaceutical active ingredient to a subject in need thereof.

“Homogeneous suspension” refers to a suspension where a substantially insoluble active agent is evenly distributed throughout the entire composition to ensure that an accurate dose is delivered to the subject.

“Liquid dosage forms” may nonexclusively include emulsions, solutions, suspensions or elixirs, where one or more of the active ingredients is dissolved, partially dissolved or in an undissolved or suspended state.

“Oral suspension” relates to a suspension that is orally ingested by a subject for therapeutic effect.

“Particle(s)” refers to a crystal, a granule, an agglomerate, or any undissolved solid material.

“Pharmaceutical suspension” refers to a suspension where at least one active pharmaceutical ingredient is present substantially in the form of undissolved solid particles, i.e., the substantially insoluble active agent. However, in an example, a portion of such active agent may be in the dissolved state. In formulating such systems, it is advantageous to minimize the amount of drug present in the dissolved state. Minimizing the amount of active agent in solution is advantageous for both the taste and chemical and physical stability of the product.

“Safe and effective amount” means an amount sufficient to induce the desired effect, but low enough to avoid serious side effects. The safe and effective amount of the compound, extract, or composition will vary with, e.g., the age, weight, health and environmental exposure of the end user, the duration and nature of the treatment, the specific extract, ingredient, or composition employed, the particular carrier utilized, and like factors.

“Subject,” “patient,” or “consumer” refers to a human or animal receiving an oral suspension.

“Substantially insoluble active agent” refers to the at least one active ingredient that is present substantially in the form of undissolved solid particles.

“Suspension” refers to a two-phase system having solid substantially insoluble active agent particles dispersed throughout liquid medium. A suspension does not encompass emulsions, which are meant to describe liquids suspended within liquid carriers or syrup formulations containing only substantially fully dissolved pharmaceutical active agents.

“Therapeutic effect,” means any effect or action of an active ingredient intended to diagnose, treat, cure, mitigate, or prevent disease, or affect the structure or any function of the body.

Unless otherwise indicated, percentages used to express amounts of ingredients are percentage by weight (referred to as “weight %,” “wt %”, “% by weight” or “% (w/volume)”). Similarly, weight ratios used to express relative proportions of ingredients are also determined using percentage by weight (i.e., weight ratios are calculated by dividing the percentage by weight of one ingredient by another).

Unless stated otherwise, all ranges are inclusive of the endpoints, e.g., “from 4 to 9” includes the endpoints 4 and 9.

As used herein, the term “about” refers to within 5% weight, within 4% weight, within 3% weight, within 2.5% weight, within 2% weight, or within 1% weight of a disclosed value.

The present invention provides a new methodology to evaluate the homogeneity of oral suspensions to ensure that such oral suspensions, especially pharmaceutical suspensions, will be safe and effective for consumer use. Preferably, pharmaceutical suspensions are homogenous suspensions to ensure that a subject receives an approved dose of an active agent. However, in the case where a pharmaceutical suspension is not completely homogenous, the instructions for use may include an instruction for the subject to “shake well” before use.

According to an example, the method for evaluating homogeneity of a suspension containing an active agent may include the steps of: (a) obtaining a test sample of the suspension in a container; (b) subjecting the container to shaking at a shaker speed and for a duration of shaking; (c) removing an aliquot of the suspension from the container; (d) measuring the amount of the active agent in the aliquot; and (e) comparing the amount of the active agent in the aliquot to an acceptance criteria.

According to an example, the container may be an enclosed beaker, a measuring cup, flask, test tube or other vessel that is routinely used in a laboratory or medical setting. According to another example, the container may identical or similar to the bottle or vessel that the suspension will be commercially sold in. It is important for the methodology to mimic actual use and shaking of the product by a subject.

According to one example, the test sample of the suspension is the entire contents or volume of the suspension as sold in the commercial bottle, for example 4 fluid ounces or 118 milliliters (mL), or alternatively 8 fluid ounces or 236 mL. This enables accurate understanding of homogeneity based on the simulation of actual or real-world use.

According to another example, the test sample of the suspension may be about 1 fluid ounce, about 1.5 fluid ounce, about 2 fluid ounces, about 2.5 fluid ounces, about 3 fluid ounces, about 3.5 fluid ounces, about 4 fluid ounces, about 4.5 fluid ounces, about 5 fluid ounces, about 5.5 fluid ounces, about 6 fluid ounces, about 7 fluid ounces, about 7.5 fluid ounces, about 8 fluid ounces, about 9 fluid ounces or about 10 fluid ounces. In another example, the test sample of the suspension may be about 30 mL, about 50 mL, about 60 mL, about 75 mL, about 100 mL, about 120 mL, about 150 mL, about 175 mL, about 200 mL, about 205 mL, about 225 mL, about 235 mL, about 250 mL, about 270 mL, about 275 mL, or about 300 mL

In one example, the shaker speed may be a low speed of about 140 rpm to about 220 rpm, or about 160 rpm to about 200 rpm, or about 170 rpm to about 190 rpm, or about 175 rpm to about 185 rpm, or about 180 rpm. In another example, the shaker speed may be a high speed of about 240 rpm to about 320 rpm, or about 260 rpm to about 300 rpm, or about 270 rpm to about 290 rpm, or about 275 rpm to about 285 rpm, or about 280 rpm. In another example, the shaker speed may be a medium speed of about 190 rpm to about 270 rpm, or about 200 rpm to about 260 rpm, or about 210 rpm to about 250 rpm, or about 220 rpm to about 240 rpm, or about 225 rpm to about 235 rpm, or about 230 rpm. In a further example, the shaker speed may include a series of two or more speeds, for example a low speed and then a high speed, or a low speed, then a medium speed and then a high speed.

According to an example, the shaker speed may be calibrated based on subject(s) understanding of shaking instructions on a particular suspension package. For example, if instructions tell a user to “shake well before use,” a subject or panel of subjects may be recorded or monitored as they follow this instruction. In one example, a video recording may be taken of subject(s) shaking well before use and the force, tempo and number of shakes may be recorded. In another example, a sensor may be attached to a test container used by the subject(s) to record the force, tempo and number of shakes. The shaker speed may then be calibrated to mimic the subject's interpretation of this instruction.

According to an example, the duration of shaking may be about 5 seconds to about 45 seconds, or about 5 seconds, or about 10 seconds, or about 15 seconds, or about 30 seconds or about 45 seconds. According to an example, the duration of shaking may be one minute or less. According to an example, the duration of shaking may be less than one minute.

Similar to the example above, the duration of shaking may be calibrated based on subject(s) understanding of shaking instructions on a particular suspension package. For example, if instructions tell a user to “shake well before use” a subject or panel of subjects may be recorded or monitored as they follow this instruction. In one example, a video recording may be taken of subject(s) shaking well before use and the duration of shaking may be recorded. In another example, a sensor may be attached to a test container used by the subject(s) to record the duration of shaking. The duration of shaking may then be calibrated to mimic the subject's interpretation of this instruction.

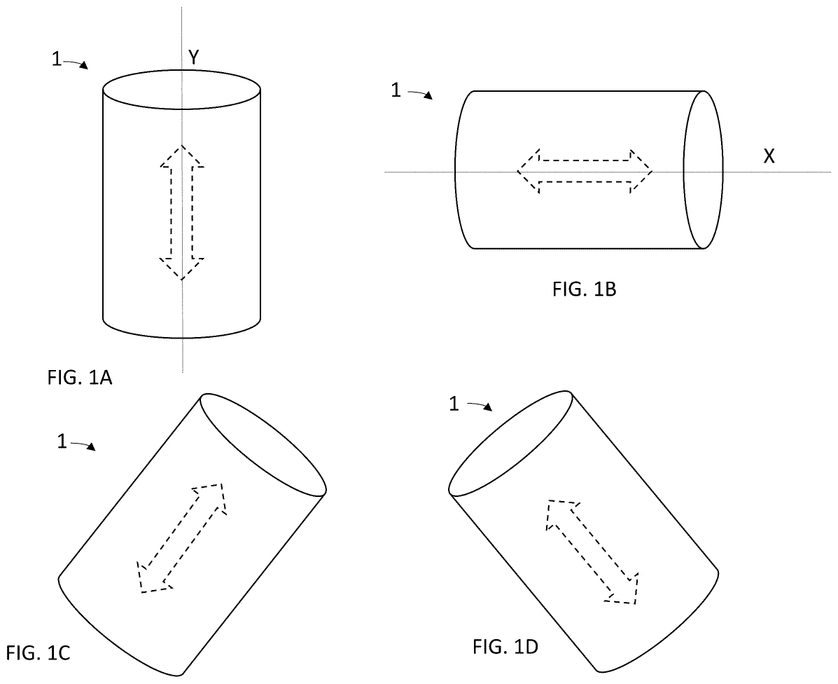

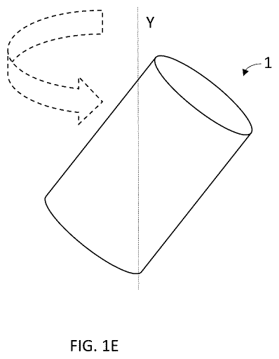

As shown in FIGS. 1A to 1D, the orientation of the container 1 and/or the direction of shaking may further be refined. For example, the container 1 may be shaken along a vertical axis Y of the container 1, as shown in Fig. A. Or, shaking may be along a horizontal axis X of the container 1, as shown in FIG. 1B. Shaking the container at an angle to vertical axis Y, as shown in FIGS. 1C and 1D, is further possible. For example, the angle may be about 10 degrees to about 80 degrees from the vertical axis, or about 40 degrees, or about 45 degrees, or about 50 degrees. The two-directional arrow in each of FIGS. 1A-1D show an exemplary back-and-forth direction of shaking.

According to an example, the step of shaking may be conducted using a fixed speed reciprocal shaker, also known as a flat bed shaker, like the Eberbach E6010.00 manufactured by Eberbach Corporation.

In an alternative example, a wrist action shaker may be used to simulate the back and forth motion of the human wrist. An example of a wrist action shaker is the Burrell Scientific Wrist Action® Model 95 Laboratory Shaker sold by Fisher Scientific. In one example, the step of shaking may include swirling the suspension in the container. As shown in FIG. 1E, during shaking, the container may be rotated or swirled around a vertical axis Y.

The orientation of the container and direction of shaking may be calibrated on test equipment based on observation of a subject(s) understanding of shaking instructions, or use instructions, on a particular suspension package. For example, if instructions tell a user to “shake well before use” a subject or panel of subjects may be recorded or monitored as they follow this instruction. In one example, a video recording may be taken of a subject shaking well before use and the orientation of the container held by the subject and direction of shaking may be recorded. In another example, a sensor may be attached to a test container used by the subject(s) to record the orientation of the container and direction of shaking. The orientation of the container and direction of shaking in the test method may then be calibrated to mimic the subject's interpretation of this instruction.

According to an example, the aliquot removed from the container after shaking may be about 1 mL, about 2.5 mL, about 5.0 mL, about 7.5 mL, about 10 mL, about 12.5 mL, about 15 mL or about 20 mL. The aliquot may correlate to the amount a subject would dispense from a bottle during use. Alternatively, the aliquot may correlate to a multiple of the dose a subject would dispense from a bottle during use. For example, if the typical dose is 5 mL, an aliquot may be dispensed at 10 mL, or another multiple of 5 mL. According to a further example, the aliquot removed from the container may represent a portion of the container, for example a first third, middle third, and last third of the bottle to evaluate the homogeneity of different portions of the container after shaking. In this example, the bottle may be divided for sampling in many different ways (e.g., in halves, thirds, fourths, fifths, etc.).

Removal of the aliquot from the container may be done in several different ways. For example, according to one example, the aliquot may be removed from the container using a pipette. The aliquot may then be transferred into a volumetric flask, or other container, for dilution and quantitative measurement.

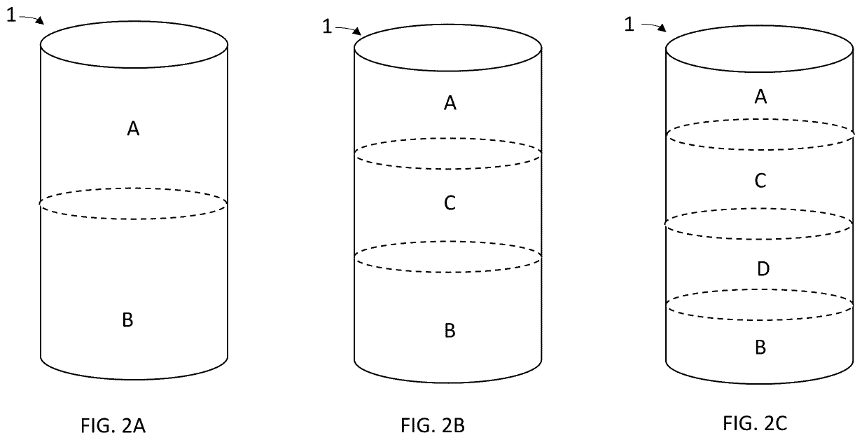

As shown in FIGS. 2A to 2C, the aliquot may be removed from different portions of the container 1 to understand how the homogeneity of the active agent within the suspension impacts the administered dose after shaking. For example, different active agents may settle towards the top or bottom of a container during storage and non-use. For this reason, it may be prudent to sample several portions of a container 1, for example one or both of a top portion or a bottom portion of the container 1. As shown in FIG. 2A, a top portion A may be the top 50% of the container 1, whereas the bottom portion B may be the bottom 50% of the container 1. Alternatively, as shown in FIG. 2B, the container 1 may be considered in thirds, where the top portion A is the top third of the container 1, the bottom portion B is the bottom third of the container 1, and a middle portion C is the middle third of the container 1. Or, as shown in FIG. 2C, the container 1 could be considered in fourths, where the top portion A is the top fourth of the container, the bottom portion B is the bottom fourth of the container, and the top middle portion C and bottom middle portion D of the container are envisioned in fourths. Other examples and divisions of the container are further contemplated.

According to another example, the aliquot may be removed from the container using a syringe or other dose dispensing device. This example may mimic certain children's or infant's oral pharmaceutical suspensions that include a syringe for subject dosing.

According to a further example, the aliquot may be poured or squeezed from the container into a dosing cup or spoon. The container may need to be inverted or tipped at an angle for removal of the aliquot. This again mimics certain children's oral pharmaceutical suspensions that include a small plastic dosing cup or spoon for administration. The dosing cup, spoon and syringe may be rinsed with diluent during the analytical procedure to ensure that all of the contents are added to the analysis.

In another example, the container may include a child-safety flow-restrictor in addition to a dosing cup, spoon and/or a syringe. In this case, the container may need to be inverted for removal of the aliquot through the flow-restrictor. The methodology aims to understand the amount of active ingredient that is administered to subjects after they have followed shaking instructions provided on the package. The more closely the test methodology is to a subject's actual use, the more accurate the results will be.

The suspension tested under the present invention preferably contains one or more active ingredients, also called active agents. According to an example, the one or more active agents are substantially insoluble. According to another example, the active agents are pharmaceutical active ingredients that may include analgesics, anti-inflammatory agents, antiarthritics, anesthetics, antihistamines, antitussives, antibiotics, anti-infective agents, antipyretics, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, cough suppressants, decongestants, expectorants, oral contraceptives, diuretics, gastrointestinal agents, migraine preparations, motion sickness products, mucolytics, muscle relaxants, osteoporosis preparations, polydimethylsiloxanes, respiratory agents, sleep-aids, urinary tract agents, and pharmaceutically acceptable salts thereof, derivatives thereof, combinations thereof and mixtures thereof.

In accordance with an example, the active ingredient may be selected from acetyl salicylic acid, acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; and pharmaceutically acceptable salts thereof, derivatives thereof, combinations thereof and mixtures thereof.

In an example, the active ingredient of the suspension may be a nonsteroidal anti-inflammatory drug (NSAID). Examples of NSAIDs include ibuprofen, naproxen, benoxaprofen, naproxen sodium, fenbufen, flurbiprofen, fenoprofen, fenbuprofen, ibuprofen, ketoprofen, indoprofen, pirprofen, carpofen, oxaprofen, pranoprofen, microprofen, tioxaprofen, suprofen, alminoprofen, tiaprofenic acid, fluprofen, bucloxic acid, celecoxib, and pharmaceutically acceptable salts thereof, derivatives thereof, combinations thereof and mixtures thereof.

According to an example, the suspension to be evaluated may comprise acetaminophen as an active agent. As used herein, the term “APAP” means acetaminophen or N-acetyl para-aminophenol, including, but not limited to, pharmaceutically acceptable salts, esters, or derivatives thereof.

Alternatively, the suspension may include cough and cold pharmaceutical active ingredients, which include antihistamines, cough suppressants, decongestants and expectorants, include, but are not limited to, bromopheniramine, carbinoxamine, acetylcysteine, guaifenesin, carbocysteine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, noscapine, clophedianol, menthol, benzonatate, ethylmorphone, codeine, acetylcysteine, carbocisteine, ambroxol, belladona alkaloids, sobrenol, guaiacol and cetirizine; and pharmaceutically acceptable salts thereof, derivatives thereof, combinations thereof and mixtures thereof.

Furthermore, the suspension may include suitable gastrointestinal agents as the active agent. Such gastrointestinal agents may include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotidine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate, loperamide and racecadotril; glycopyrrolate; antiemetics, such as ondansetron, analgesics, such as mesalamine.

In other examples, the suspension may include more than one active agent. For example, the suspension may include an NSAID and/or acetaminophen, and pharmaceutically acceptable salts thereof.

According to an example, the active agent is an active pharmaceutical ingredient (API) and the amount of the active agent in the aliquot is measured using an assay analysis of a USP monograph related to the API, where USP stands for United States Pharmacopeia. For example, if the active agent were acetaminophen, the USP guidance document on <Acetaminophen Oral Suspension>, Doc ID GUID-8690AABC-4A2A-468C-9B04-D62048F76DF7_10_en-US, may be used to provide the assay procedure to be used under the present methodology. The current edition of the USP guidance is USP-NF 2022 Issue 2, Published Feb. 1, 2022, although subsequently published guidance documents may be used. The current version of USP-NF standards deemed official by USP is enforceable by the U.S. Food and Drug Administration for medicines manufactured and marketed in the United States. USP provides standards of identity, strength, quality, and purity manufactured and marketed in the United States.

Under such USP guidance information, an applicable acceptance criteria may also be obtained. For example, the acceptance criteria may correspond to an approved dose of the API. As a non-limiting example, the above-referenced USP assay procedure for <Acetaminophen Oral Suspensions> states that the amount of acetaminophen in a suspension must be within 90.0% to 110.0% of the approved dosage amount. Thus, an acceptance criteria for an exemplary methodology may be 90.0%-110.0% of the approved APAP dose pursuant to USP guidelines. Or, in another example, the acceptance criteria may be about 92% to about 108%, or about 95% to about 105%, or about 97% to about 103%, if a more conservative approach is desired.

According to another example, the amount of the active agent in the aliquot may be measured using a methodology published by a compendial source other than USP, for example, but not limited to the International Organization for Standardization (ISO), the European Pharmacopoeia (EP), or the British Pharmacopoeia (BP). In a further example, a measurement methodology may be industry-, self- or internally-developed to suit the needs of the analysis. Likewise, an acceptance criteria may be adopted from a compendial source, or may be self- or internally-developed to suit the needs of the methodology.

According to an example, the methodology may measure something other than the amount of the active agent in the aliquot. According to an example, the methodology may be further used assess both physical and chemical stability parameters. Physical stability evaluations may include particle size, viscosity, rheology, organoleptic and color analysis of active ingredients. Chemical stability assessments may include active ingredient assay and degradation analysis. Or the methodology may measure the homogeneity of the suspension as a whole. Homogeneity measuring methods may include visual inspection, light spectroscopy, particle size analysis, light scattering, fluorescence, or viscosity sampling and/or analysis.

According to an example, the acceptance criteria may be unrelated to the approved dosage amount. For example, the acceptance criteria may relate directly to the homogeneity of the suspension itself, for example, about 60%, about 65%, about 70%, about 75%, about 80%, about 85%, about 90%, about 91%, about 92%, about 93%, about 94%, about 95%, about 96%, about 97%, about 98%, about 99% or about 100% homogeneous. Or, the acceptance criteria may relate to the consistency of light spectroscopy, particle size analysis, light scattering, fluorescence or viscosity measurements.

The present invention may further involve an iterative approach to testing. For example, the methodology may include several aliquots or samples from the same suspension container to be tested for homogeneity of the active agent. Or, the methodology may include several aliquots or samples from different suspension containers of the same product to be tested.

In an example, the method may further include the steps of: (f) subjecting a remaining test sample of the suspension to additional shaking at the shaker speed and for a second duration of shaking; (g) removing a second aliquot of the suspension from the container; (h) measuring the amount of the active agent in the second aliquot; and (i) comparing the amount of the active agent in the second aliquot to a second acceptance criteria. The same process may be followed for third, fourth, fifth, etc. aliquots, until the container no longer contains any suspension fluid. Such additional steps may use the same or a different acceptance criteria from the first acceptance criteria used.

The methodologies of the present invention may be further paired with any other testing to understand the safety and efficacy of suspension products with active agents.

The methodologies of the present invention may be used during the product development process to formulate safe and efficacious products, or as a stability program to verify stability, safety and efficacy of new products and/or existing products on the market. The methodologies may be used, for example as part of a consumer testing protocol, to verify that product instructions for product use, for example instructions related to shaking, are accurate, understandable and reproducible by consumers. The methodologies may be used to ensure stability and quality control of suspension products. The methodologies may be used to compare the homogeneity of two different suspension products or formulations. The methodologies may even be used to show consumers the safety and efficacy of suspension products through a marketing demo or the like. The methodologies may also be used as a standard to verify safety and efficacy of suspension products or formulations, or as part of a procedure to verify other USP standards.

### EXAMPLES

The following examples set forth below further illustrate examples of the present invention. The present invention is not limited to the examples contained therein.

### Example 1: Test Set 1

### Test Formulation A

Samples for Test Formulation A are based on a commercially available product, which was purchased for testing purposes. The ingredient list for Test Formulation A includes 160 mg of acetaminophen (in each 5 mL dose), as well as inactive ingredients including organic agave syrup, organic blueberry flavor, natural citrus extract, non-GMO flavor and purified water. The label instructions for use of Test Formulation A are to “shake well before using.”

### Test Formulation B

Samples for the following Test Formulation B are development formulations made as lab scale batches for execution of this protocol. Reference Table 1 for a description of the formulation to be assessed.

TABLE 1
Description of Test Formulation B to be Evaluated
FORMULA RECORD

| | Unit | Weight |
|---|---|---|
| | Weight | Percent |
| Ingredient | mg/5 mL | (w/v) |
| Purified Water USP | 1000.0 | 20.0 |
| Sorbitol Solution USP | 1000.0 | 20.0 |
| Microcrystalline Cellulose and | 35.0 | 0.70 |
| Carboxymethylcellulose Sodium | | |
| NF (Avicel ® RC-591) | | |
| Xanthan Gum NF (Xantural ® 180) | 7.0 | 0.14 |
| High Fructose Corn Syrup NF 42% | 3591.9 | 71.84 |
| Glycerin USP | 500.0 | 10.0 |
| Anhydrous Citric Acid USP | 5.75 | 0.115 |
| Sodium Benzoate NF | 10.0 | 0.20 |
| Acetaminophen USP or | 160.0 | 3.2 |
| Experimental Acetaminophen USP | | |
| Sucralose 25% Liquid Concentrate | 10.0 | 0.20 |
| Artificial Grape Flavor # 5-12335 | 2.50 | 0.05 |
| Artificial Grape Flavor 597303 C | 3.00 | 0.06 |
| D&C Red No. 33 Certified | 0.05 | 0.0010 |
| FD&C Blue No. 1 Certified | 0.0075 | 0.00015 |

### Experimental Parameters—Part 1

Test samples of Formulations A and B may be analyzed for assay as per USP monograph <Acetaminophen Oral Suspension>. Test samples are to be prepared one at a time to prevent unnecessary settling of solution prior to sampling. Test samples will be dosed immediately after shaking is completed. As an exemplary test methodology, the flatbed shaker may be set to the appropriate setting for the specified time points, according to Table 2.

TABLE 2
Samples to be Analyzed

| | Bottle | Shaking | Shaking |
|---|---|---|---|
| Sample | Number | Time | Speed |
| Test | 1 | 15 seconds | High |
| Formula A | 2 | 30 seconds | Low |
| Test | 3 | 15 seconds | High |
| Formula B | 4 | 30 seconds | Low |

### Sample Preparation—Part 1

Test samples of APAP oral suspensions, namely Test Formulations A and B, are to be prepared one at a time, in the following manner:
a. Lay unopened commercial bottle flat on its side, on the flatbed shaker. Secure with foam blocks, or other similar items, to ensure that the bottle does not move.
b. Turn on the shaker to the appropriate speed and time of shaking using a calibrated timer.
c. Once the appropriate time is reached, immediately turn off the shaker and remove the bottle.
d. Immediately following shaking, tare the dosing cup provided with each bottle on the balance. Dispense 5 mL of the product (i.e., the aliquot) into the tared dosing cup and record the weight.
e. Transfer the contents of the dosing cup into a 200 mL volumetric flask. Using diluent, rinse the dosing cup containing all remaining aliquot into the flask, making sure not to exceed total flask volume of 200 mL.
f. The remainder of sample preparation and analysis is to be performed according to USP guidelines. In some cases, such as a study that uses a commercial children's or adult dosing cup, the sample preparation may need to be slightly modified from USP guidelines to adjust to the approved dose amount.

### Data Analysis Methods and Acceptance Criteria

The sample preparation described above is to be analyzed using chromatographic parameters described in the assay analysis of the USP monograph for <Acetaminophen Oral Suspension>. The various data points acquired from Test Formulation A, a commercially available product that is marketed as having “clean” ingredients, were compared with the data generated from Test Formulation B, which is similar to commercially available Children's acetaminophen liquid suspensions, and are discussed below.

### Results

Test Formulations A and B were subjected to various tests under different examples of the present methodology, including the Experimental Parameters above. The term “Shaker Speed” refers to the revolutions per minute (rpm) of shaking that the test sample is subjected to. For this experiment and as listed in Table 1, the shaker speed of “Low” was 180 revolutions per minute (rpm) and the shaker speed of “High” was 280 rpm. The shaker equipment used was flat bed shaker, like the Eberbach E6010.00 manufactured by Eberbach Corporation. Additionally, one test used an “aggressive hand shaken” method which relied on a human tester hand shaking the test sample until visual confirmation of homogeneity. The term “Duration of Shaking” refers to the amount of time the test sample was subjected to shaking at the Shaker Speed. This test evaluated the accuracy of dosing when administering each of Formulations A and B based on variations in product shaking time and speed.

TABLE 3
Homogeneity Results from Shaken Suspension Formulations

| | Formulation | Duration | Shaker | Sampled | APAP |
|---|---|---|---|---|---|
| Test # | A or B | of Shaking | Speed | Region | (%) |
| 1 | A | 5 seconds | Low | Top | 117.1 |
| | | | | Bottom | 39.1 |
| 2 | A | 15 seconds | Low | Top | 159.8 |
| | | | | Bottom | 41.2 |
| 3 | A | 30 seconds | High | Top | 113.5 |
| | | | | Bottom | 73.2 |
| | | | Low | Top | 415.2 |
| | | | | Bottom | 37.7 |
| 4 | A | 45 seconds | High | Top | 115.2 |
| | | | | Bottom | 72.2 |
| | | | Low | Top | 222.4 |
| | | | | Bottom | 32.5 |
| 5 | A | 10 seconds | Aggressive | Top | 101.5 |
| | | | Hand Shaken | Bottom | 97.4 |
| 6 | B | 15 seconds | Low | Top | 89.8 |
| | | | Low | Bottom | 94.8 |
| 7 | B | 45 seconds | Low | Top | 92.8 |
| | | | Low | Bottom | 94.7 |

After each test sample was shaken at the Shaker Speed for the listed Duration of Shaking (e.g., 5-45 seconds), the tester took aliquots of each formulation from the top of the bottle and the bottom of the bottle using a pipette. These aliquots were tested for the percentage of acetaminophen (APAP) present, where the percentage was based on the amount of expected dose. In this example, the expected dose is the approved dose of APAP, as presented on commercial suspension labels; for example, 160 mg of APAP per 5 mL. Thus, 100% APAP would denote that the expected dose of 160 mg of APAP per 5 mL was present, whereas 120% APAP would denote that 20% more APAP was present than the expected dose. The selected acceptance criteria in this case was 90.0%-110.0% of the approved APAP dose pursuant to USP guidelines for <Acetaminophen Oral Suspension>.

As seen in Table 3, Formulation B delivered results within the acceptance criteria. All results were within about 90.0% to 110.0% of the approved APAP dose.

Formulation A, however, produced varied results, all of which were outside the acceptance criteria, using the flat bed shaker depending on the duration of shaking, the shaker speed and the sampled region.

For example, results from the top sampled region of Formulation A, using the flat bed shaker, were consistently above the 110.0% acceptance criteria. This indicates that homogeneity was not achievable with the duration of shaking and the shaker speeds tested. In an extreme example, in Test #3 at a shaker duration of 30 seconds and shaker speed of “low,” the APAP percentage was 415.2%, which is over four times the recommended dose. Even when a “high” shaking speed was used, Formulation A's APAP percentages were above the 110.0% acceptance criteria. These results may indicate that Formulation A needs to be reformulated to be more homogeneous or that Formulation A's shaking instructions need to be revised to achieve homogeneity within the acceptance criteria. In particular, these results may indicate that there is a risk that a subject could receive a higher APAP dose than recommended if pouring a dose from the top portion of a bottle.

The results from the bottom sampled region of Formulation A, using the flat bed shaker, were consistently below the 90.0% acceptance criteria. This indicates that homogeneity was not achievable with the duration of shaking and the shaker speeds tested. In Test #3, for example, at a shaker duration of 30 seconds and shaker speed of “low,” the APAP percentage was 37.7%, which is less than half of recommended dose. Even when a “high” shaking speed was used, Formulation A's APAP percentages were less than 75% of the approved dose. These results may indicate that Formulation A needs to be reformulated to be more homogenous or that Formulation A's shaking instructions need to be revised to achieve homogeneity within the acceptance criteria. In particular, these results may indicate that there is a risk that a subject could receive a lower APAP dose than recommended, especially if higher than expected APAP amounts were previously dispensed from the top of the bottle. This could translate to a subject not receiving the expected therapeutic effect from taking the medication.

Formulation A produced the best results when an “Aggressive Hand Shaken” method was used which, as described above, relied on a human tester hand shaking the test sample until visual confirmation of homogeneity. In this case, the homogeneity of the APAP was within the 90.0% to 110.0% acceptance criteria. This may indicate that visual inspection of homogeneity should be included as part of the “shake well before using” instruction. Or, perhaps, the aggressive hand shaking was a longer duration of shaking and/or a higher shaker speed than that tested using the flat bed shaker. As a future step to further improve the present test methodology, it is recommended that such aggressive hand shaking to achieve visual homogeneity be recorded and analyzed, so that the duration of shaking and shaking speed can be better understood and possibly incorporated into the methodology for the flat bed shaker.

### Example 2: Test Set 2

The Test formulations from Example 1 were tested using an alternative exemplary set of parameters as described below:

Samples were analyzed using the procedure in Table 4. Three (3) separate, new unopened bottles were used for evaluation of each shaking time (5, 10, &15 seconds) and shaking speed (Low-180 oscillations/min & High-280 oscillations/min). Shaking time was documented using a calibrated timer and shaking speed was preset on the flatbed shaker (model Eberbach 6010). Three (3) doses were extracted from each bottle (with shaking performed in between each dose); the sequential doses were prepared at least 4-hours apart as the label instructs.

Samples were prepared one at a time to prevent unnecessary settling of the product. Each bottle was secured horizontally on a flatbed shaker for the designated time and speed. Immediately after the shaking was completed, a 5 mL dose of the product was dispensed into the dosing cup provided with the product and weighed. The samples were then diluted to a final concentration of 0.01 mg/mL APAP, in accordance with the USP monograph <Acetaminophen Oral Suspension>.

The samples were quantified against an APAP reference standard (0.01 mg/mL). Analysis was performed by reversed phase high-performance liquid chromatography (HPLC), with instrument parameters in accordance with the USP monograph. All USP system suitability requirements listed in Table 3 were applied and met. The APAP assay label claim percentage was calculated for all samples and compared.

### Sample Preparation Steps—Part 2

1. Samples were prepared one at a time to prevent unnecessary settling of suspension prior to sampling.
2. Samples were poured into the dosing cup immediately after shaking was completed.
3. The flatbed shaker was set to the appropriate setting for the specified time points, according to Table 2.
4. Three bottles were prepared for each shake time/shake speed (i.e., 3-bottles for 5-seconds low speed and 3-bottles for 5-seconds high speed; total 18-bottles per product)
5. Three samples were prepared for each bottle (dispense 3-doses, rinsing dosing cup with diluent in between each dose). Sequential doses were performed after at least four hours of sitting on bench top to mimic at-home dosing. The bottles were shaken again for a specified time between each dose.

TABLE 4
Analyzed Samples

| | | Shaking Time |
|---|---|---|
| Product | Test # | (Low Speed & High Speed) |
| Test | 1 | 5 seconds |
| Formula A | 2 | 15 seconds |
| | 3 | 30 seconds |
| Test | 1 | 5 seconds |
| Formula B | 2 | 15 seconds |
| | 3 | 30 seconds |

1. The bottle was laid flat on its side, on the flatbed shaker. The bottle was secured with foam blocks to ensure bottle did not move.
2. The shaker was turned on to the appropriate speed and the shaking was timed using a calibrated timer.
3. Once the appropriate time was reached, the shaker was immediately turned off and the sample was removed.
4. Immediately following shaking, the dosing cup provided with each bottle was tared on the balance. 5 mL of the product was dispensed into the tared dosing cup and the weight was recorded.
5. The contents of the dosing cup were transferred into a 200 mL volumetric flask (˜0.8 mg/mL). Using diluent, all remaining sample was rinsed into the flask, making sure not to exceed total flask volume of 100 mL. The contents of the flask were swirled to visually confirm sample was dissolved. The sample was diluted to volume using the diluent.
6. The remainder of sample preparation and analysis was performed according to USP monograph <Acetaminophen Oral Suspension>.

### Sample Results—Part 2

### Acceptance Criteria

For all samples, the assay result acceptance criteria must be 90.0%-110.0%. This requirement is established in the USP monograph <Acetaminophen Oral Suspension>, and all monograph products must meet this threshold.

### Assay Results

TABLE 5
5-Second, Low Speed Shaking Results

| | | Acetaminophen Assay (% Label Claim) | | | |
|---|---|---|---|---|---|
| Sample | | | Bottle | | Bottle |
| Identi- | Sample | Test | Mean % & | Test | Mean % & |
| fication | # | Formula A | % RSD | Formula B | % RSD |
| Bottle 1 | 1 | 32.8 | Mean: | 97.1 | Mean: |
| Dose 1 | | | 48.6 | | 97.6 |
| Bottle 1 | 2 | 48.1 | % RSD: | 97.1 | % RSD: |
| Dose 2 | | | 33.1 | | 0.9 |
| Bottle 1 | 3 | 65.0 | | 98.7 | |
| Dose 3 | | | | | |
| Bottle 2 | 4 | 37.5 | Mean: | 98.0 | Mean: |
| Dose 1 | | | 90.2 | | 97.4 |
| Bottle 2 | 5 | 98.5 | % RSD: | 97.3 | % RSD: |
| Dose 2 | | | 54.4 | | 0.6 |
| Bottle 2 | 6 | 134.5 | | 96.8 | |
| Dose 3 | | | | | |
| Bottle 3 | 7 | 35.2 | Mean: | 98.4 | Mean: |
| Dose 1 | | | 96.7% | | 99.4 |
| Bottle 3 | 8 | 42.5 | RSD: | 101.0 | % RSD: |
| Dose 2 | | | 103.7 | | 1.4 |
| Bottle 3 | 9 | 212.4 | | 98.7 | |
| Dose 3 | | | | | |

| | | |
|---|---|---|
| Sample Mean % & | Mean: 78.5 | Mean: 98.1 |
| % RSD | % RSD: 77.4 | % RSD: 1.3 |

TABLE 6
5-Second, High Speed Shaking Results

| | | Acetaminophen Assay (% Label Claim) | | | |
|---|---|---|---|---|---|
| Sample | | | Bottle | | Bottle |
| Identi- | Sample | Test | Mean % & | Test | Mean % & |
| fication | # | Formula A | % RSD | Formula B | % RSD |
| Bottle 1 | 1 | 40.4 | Mean: 92.8 | 97.8 | Mean: 98.5 |
| Dose 1 | | | % RSD: 61.8 | | % RSD: 0.8 |
| Bottle 1 | 2 | 154.0 | | 99.3 | |
| Dose 2 | | | | | |
| Bottle 1 | 3 | 83.9 | | 98.5 | |
| Dose 3 | | | | | |
| Bottle 2 | 4 | 32.6 | Mean: 67.0 | 98.0 | Mean: 97.9 |
| Dose 1 | | | % RSD: 44.7 | | % RSD: 0.1 |
| Bottle 2 | 5 | 81.1 | | 97.8 | |
| Dose 2 | | | | | |
| Bottle 2 | 6 | 87.3 | | 97.9 | |
| Dose 3 | | | | | |
| Bottle 3 | 7 | 36.0 | Mean: 65.5 | 97.6 | Mean: 97.8 |
| Dose 1 | | | % RSD: 44.2 | | % RSD: 0.4 |
| Bottle 3 | 8 | 66.7 | | 98.2 | |
| Dose 2 | | | | | |
| Bottle 3 | 9 | 93.9 | | 97.5 | |
| Dose 3 | | | | | |

| | | |
|---|---|---|
| Sample Mean % & | Mean: 75.1 | Mean: 98.1 |
| % RSD | % RSD: 50.4 | % RSD: 0.6 |

TABLE 7
15-Second, Low Speed Shaking Results

| | | Acetaminophen Assay (% Label Claim) | | | |
|---|---|---|---|---|---|
| Sample | | | Bottle | | Bottle |
| Identi- | Sample | Test | Mean % & | Test | Mean % & |
| fication | # | Formula A | % RSD | Formula B | % RSD |
| Bottle 1 | 1 | 97.6 | Mean: 64.9 | 98.9 | Mean: 98.4 |
| Dose 1 | | | % RSD: 47.0 | | % RSD: 0.5 |
| Bottle 1 | 2 | 37.1 | | 98.3 | |
| Dose 2 | | | | | |
| Bottle 1 | 3 | 60.1 | | 97.9 | |
| Dose 3 | | | | | |
| Bottle 2 | 4 | 38.9 | Mean: 54.7 | 99.2 | Mean: 98.6 |
| Dose 1 | | | % RSD: 63.0 | | % RSD: 0.8 |
| Bottle 2 | 5 | 31.0 | | 98.9 | |
| Dose 2 | | | | | |
| Bottle 2 | 6 | 94.3 | | 97.7 | |
| Dose 3 | | | | | |
| Bottle 3 | 7 | 61.7 | Mean: 61.4 | 99.1 | Mean: 98.7 |
| Dose 1 | | | % RSD: 12.6 | | % RSD: 0.4 |
| Bottle 3 | 8 | 68.9 | | 98.3 | |
| Dose 2 | | | | | |
| Bottle 3 | 9 | 53.5 | | 98.6 | |
| Dose 3 | | | | | |

| | | |
|---|---|---|
| Sample Mean % & | Mean: 60.3 | Mean: 98.5 |
| % RSD | % RSD: 39.4 | % RSD: 0.5 |

TABLE 8
15-Second, High Speed Shaking Results

| | | Acetaminophen Assay (% Label Claim) | | | |
|---|---|---|---|---|---|
| Sample | | | Bottle | | Bottle |
| Identi- | Sample | Test | Mean % & | Test | Mean % & |
| fication | # | Formula A | % RSD | Formula B | % RSD |
| Bottle 1 | 1 | 58.7 | Mean: 53.6 | 98.4 | Mean: 98.7 |
| Dose 1 | | | % RSD: 17.0 | | % RSD: 1.1 |
| Bottle 1 | 2 | 43.1 | | 99.9 | |
| Dose 2 | | | | | |
| Bottle 1 | 3 | 59.0 | | 97.7 | |
| Dose 3 | | | | | |
| Bottle 2 | 4 | 77.1 | Mean: 63.1 | 99.2 | Mean: 98.9 |
| Dose 1 | | | % RSD: 28.4 | | % RSD: 0.8 |
| Bottle 2 | 5 | 42.9 | | 99.6 | |
| Dose 2 | | | | | |
| Bottle 2 | 6 | 69.3 | | 98.0 | |
| Dose 3 | | | | | |
| Bottle 3 | 7 | 34.2 | Mean: 48.1 | 98.2 | Mean: 98.5 |
| Dose 1 | | | % RSD: 34.8 | | % RSD: 0.3 |
| Bottle 3 | 8 | 43.5 | | 98.6 | |
| Dose 2 | | | | | |
| Bottle 3 | 9 | 66.7 | | 98.8 | |
| Dose 3 | | | | | |

| | | |
|---|---|---|
| Sample Mean % & | Mean: 54.9 | Mean: 98.7 |
| % RSD | % RSD: 26.6 | % RSD: 0.7 |

TABLE 9
30-Second, Low Speed Shaking Results

| | | Acetaminophen Assay (% Label Claim) | | | |
|---|---|---|---|---|---|
| Sample | | | Bottle | | Bottle |
| Identi- | Sample | Test | Mean % & | Test | Mean % & |
| fication | # | Formula A | % RSD | Formula B | % RSD |
| Bottle 1 | 1 | 39.1 | Mean: 64.7 | 97.9 | Mean: 98.1 |
| Dose 1 | | | % RSD: 39.9 | | % RSD: 0.6 |
| Bottle 1 | 2 | 64.3 | | 98.7 | |
| Dose 2 | | | | | |
| Bottle 1 | 3 | 90.8 | | 97.6 | |
| Dose 3 | | | | | |
| Bottle 2 | 4 | 31.6 | Mean: 83.0 | 98.9 | Mean: 98.4 |
| Dose 1 | | | % RSD: 58.2 | | % RSD: 0.6 |
| Bottle 2 | 5 | 127.4 | | 98.4 | |
| Dose 2 | | | | | |
| Bottle 2 | 6 | 90.1 | | 97.8 | |
| Dose 3 | | | | | |
| Bottle 3 | 7 | 26.9 | Mean: 52.2 | 98.5 | Mean: 97.9 |
| Dose 1 | | | % RSD: 43.0 | | % RSD: 0.6 |
| Bottle 3 | 8 | 60.0 | | 97.9 | |
| Dose 2 | | | | | |
| Bottle 3 | 9 | 69.7 | | 97.4 | |
| Dose 3 | | | | | |

| | | |
|---|---|---|
| Sample Mean % & | Mean: 66.7 | Mean: 98.1 |
| % RSD | % RSD: 48.8 | % RSD: 0.5 |

TABLE 10
30-Second, High Speed Shaking Results

| | | Acetaminophen Assay (% Label Claim) | | | |
|---|---|---|---|---|---|
| Sample | | | Bottle | | Bottle |
| Identi- | Sample | Test | Mean % & | Test | Mean % & |
| fication | # | Formula A | % RSD | Formula B | % RSD |
| Bottle 1 | 1 | 39.1 | Mean: 70.0 | 98.6 | Mean: 98.9 |
| Dose 1 | | | % RSD: 45.4 | | % RSD: 0.3 |
| Bottle 1 | 2 | 68.2 | | 99.1 | |
| Dose 2 | | | | | |
| Bottle 1 | 3 | 102.6 | | 98.9 | |
| Dose 3 | | | | | |
| Bottle 2 | 4 | 41.8 | Mean: 65.3 | 99.0 | Mean: 98.7 |
| Dose 1 | | | % RSD: 39.2 | | % RSD: 0.6 |
| Bottle 2 | 5 | 61.5 | | 99.1 | |
| Dose 2 | | | | | |
| Bottle 2 | 6 | 92.5 | | 98.1 | |
| Dose 3 | | | | | |
| Bottle 3 | 7 | 44.2 | Mean: 69.8 | 98.9 | Mean: 98.6 |
| Dose 1 | | | % RSD: 36.3 | | % RSD: 1.2 |
| Bottle 3 | 8 | 70.4 | | 99.5 | |
| Dose 2 | | | | | |
| Bottle 3 | 9 | 94.8 | | 97.3 | |
| Dose 3 | | | | | |

| | | |
|---|---|---|
| Sample Mean % & | Mean: 68.3 | Mean: 98.7 |
| % RSD | % RSD: 35.3 | % RSD: 0.7 |

TABLE 11
Summary of Results for Test Formula A

| | | | | # Of | # Of |
|---|---|---|---|---|---|
| Shaking | Shaking | | Acceptance | Samples | Samples |
| Time | Speed | Product | Criteria | Passed | Failed |
| 5 | Low: | Test | 90.0-110.0% | 1 | 8 |
| | 180 | Formula A | | | |
| | oscillations/min | | | | |
| 5 | High: | Test | 90.0-110.0% | 1 | 8 |
| | 280 | Formula A | | | |
| | oscillations/min | | | | |
| 15 | Low: | Test | 90.0-110.0% | 2 | 7 |
| | 180 | Formula A | | | |
| | oscillations/min | | | | |
| 15 | High: | Test | 90.0-110.0% | 0 | 9 |
| | 280 | Formula A | | | |
| | oscillations/min | | | | |
| 30 | Low: | Test | 90.0-110.0% | 2 | 7 |
| | 180 | Formula A | | | |
| | oscillations/min | | | | |
| 30 | High: | Test | 90.0-110.0% | 3 | 6 |
| | 280 | Formula A | | | |
| | oscillations/min | | | | |

TABLE 12
Summary of Results for Test Formula B

| | | | | # Of | # Of |
|---|---|---|---|---|---|
| Shaking | Shaking | | Acceptance | Samples | Samples |
| Time | Speed | Product | Criteria | Passed | Failed |
| 5 | Low: | Test | 90.0-110.0% | 9 | 0 |
| | 180 | Formula B | | | |
| | oscillations/min | | | | |
| 5 | High: | Test | 90.0-110.0% | 9 | 0 |
| | 280 | Formula B | | | |
| | oscillations/min | | | | |
| 15 | Low: | Test | 90.0-110.0% | 9 | 0 |
| | 180 | Formula B | | | |
| | oscillations/min | | | | |
| 15 | High: | Test | 90.0-110.0% | 9 | 0 |
| | 280 | Formula B | | | |
| | oscillations/min | | | | |
| 30 | Low: | Test | 90.0-110.0% | 9 | 0 |
| | 180 | Formula B | | | |
| | oscillations/min | | | | |
| 30 | High: | Test | 90.0-110.0% | 9 | 0 |
| | 280 | Formula B | | | |
| | oscillations/min | | | | |

In this study, Test Formula B had consistent product uniformity and dosing accuracy, as all samples met the USP criteria regardless of shaking time or speed. Sample results ranged from 96.8%-101.0% Label Claim, which is well within the USP monograph criteria of 90.0-110.0%. The low % RSD values for each of the Test Formula B bottles further demonstrate homogeneity with low variability throughout the bottle. The Test Formula B products that were evaluated within this report were consistently well within the USP monograph acceptance criteria of 90.0-110.0%, with minimal variability.

Alternatively, the Test Formula A products that were evaluated in the study had increased variability with product uniformity and dosing accuracy. A majority of the samples analyzed did not meet the USP criteria of 90.0-110.0% (as reported in Table 11), with sample results ranging from 26.9%-212.4% Label Claim. The high % RSD values for each of the Test Formula A bottles further demonstrated the high variability throughout the bottle. Overall, the variation in shaking time and speed had no effect on the homogeneity of the product.

These results show the importance of the exemplary methodologies described herein to evaluate the homogeneity of oral suspensions to ensure that such oral suspensions, especially pharmaceutical suspensions, will be safe and effective for consumer use. Such methodologies may be used during the product development process, and may further be used to verify that product instructions for product use are accurate and reproducible. Such methodologies may also be used for quality control purposes, or even as part of a marketing demonstration or consumer testing protocol. These methodologies may enable the safe development of pharmaceutical suspension products with clean ingredients.

While systems and methods have been described in connection with the various examples of the various figures and description, it will be appreciated by those skilled in the art that changes could be made to the examples without departing from the broad inventive concept thereof. It is understood, therefore, that this disclosure is not limited to the particular examples disclosed, and it is intended to cover modifications within the spirit and scope of the present disclosure as defined by the claims.

It is to be appreciated that certain features of the invention which are, for clarity, described herein in the context of separate examples, may also be provided in combination in a single example. That is, unless obviously incompatible or specifically excluded, each individual example is deemed to be combinable with any other example(s) and such a combination is considered to be another example. Conversely, various features of the invention that are, for brevity, described in the context of a single example, may also be provided separately or in any sub-combination. Finally, while an example may be described as part of a series of steps or part of a more general structure, each said step may also be considered an independent example in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various examples of the present invention. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

1. A method for evaluating homogeneity of a suspension containing an active agent, comprising:
(a) obtaining a test sample of the suspension in a container;
(b) subjecting the container to shaking at a shaker speed and for a duration of shaking;
(c) removing an aliquot of the suspension from the container;
(d) measuring the amount of the active agent in the aliquot; and
(e) comparing the amount of the active agent in the aliquot to an acceptance criteria.

2. The method of claim 1, wherein the shaker speed comprises a low speed of about 140 rpm to about 220 rpm, or about 160 rpm to about 200 rpm, or about 170 rpm to about 190 rpm, or about 175 rpm to about 185 rpm, or about 180 rpm.

3. The method of claim 1, wherein the shaker speed comprises a high speed of about 240 rpm to about 320 rpm, or about 260 rpm to about 300 rpm, or about 270 rpm to about 290 rpm, or about 275 rpm to about 285 rpm, or about 280 rpm.

4. The method of claim 1, wherein the duration of shaking is about 5 seconds to about 45 seconds, or about 5 seconds, or about 10 seconds, or about 15 seconds, or about 30 seconds or about 45 seconds.

5. The method of claim 1, wherein step (b) further comprises shaking the container along a vertical axis.

6. The method of claim 1, wherein step (b) further comprises shaking the container along a horizontal axis.

7. The method of claim 1, wherein step (b) further comprises shaking the container at an angle to a vertical axis.

8. The method of claim 7, wherein the angle is about 10 degrees to about 80 degrees from the vertical axis.

9. The method of claim 1, wherein step (b) is conducted using a fixed speed reciprocal shaker.

10. The method of claim 1, wherein the test sample is about 4 or 8 fluid ounces.

11. The method of claim 1, wherein the aliquot of step (c) is removed using a pipette.

12. The method of claim 1, wherein the aliquot of step (c) is removed from a top portion of the container.

13. The method of claim 1, wherein the aliquot of step (c) is removed from a bottom portion of the container.

14. The method of claim 1, wherein the aliquot of step (c) is removed from a middle portion of the container.

15. The method of claim 1, wherein the aliquot of step (c) is removed using a syringe.

16. The method of claim 15, wherein the container must be inverted prior to removal of the aliquot in step (c).

17. The method of claim 1, wherein the aliquot of step (c) is poured or squeezed from the container into a dosing cup.

18. The method of claim 1, wherein the active agent is substantially insoluble.

19. The method of claim 1, wherein the active agent is an active pharmaceutical ingredient (API).

20. The method claim 19, wherein the amount of the active agent in the aliquot is measured in step (d) using an assay analysis of a USP monograph related to the API.

21. The method of claim 19, wherein the acceptance criteria in step (e) corresponds to an approved dose of the API.

22. The method of claim 1, further comprising:
(f) subjecting a remaining test sample of the suspension in the container to additional shaking at the shaker speed and for a second duration of shaking;
(g) removing a second aliquot of the suspension from the container;
(h) measuring the amount of the active agent in the second aliquot; and
(i) comparing the amount of the active agent in the second aliquot to a second acceptance criteria.

23. The method of claim 22, wherein the second acceptance criteria is the same as the acceptance criteria.

24. The method of claim 22, wherein the second acceptance criteria is different from the acceptance criteria.

25. The method of claim 1, wherein the step of subjecting the container to shaking at the shaker speed and for the duration of shaking corresponds to a user instruction associated with the test sample.

26. The method of claim 25, wherein the user instruction is provided on a commercial package of the test sample.

27. The method of claim 26, wherein the user instruction is to “shake well.”

28. The method of claim 1, wherein the step of subjecting the container to shaking at the shaker speed and for the duration of shaking corresponds to an observation of a user based on the user's interaction with a user instruction associated with the test sample.

29. The method of claim 28, wherein the user instruction is provided on a commercial package of the test sample.

30. The method of claim 29, wherein the user instruction is to “shake well.”

31. The method of claim 28, wherein the observation comprises the user's shaker speed and duration of shaking.
